(19) United States
(12) Patent Application Publication
(10) Pub. No.: US 2018/0077899 A1
(43) Pub. Date: Mar. 22, 2018

(54) Methods and Compositions for Dag Mitigation

(71) Applicant: Empire Technology Development LLC, Wilmington, DE (US)

(72) Inventor: Georgius Abidal Adam, Edensor Park (AU)

(21) Appl. No.: 15/272,914

(22) Filed: Sep. 22, 2016

(51) Int. Cl. A01K 13/00; A61K 8/49; A61K 8/41; A61Q 5/02

(52) U.S. Cl. CPC A01K 13/00; A61K 8/4973; A61K 8/411; A61Q 5/02; A61K 2800/59; A61K 2800/882; A61K 2800/95; A61K 2800/81; A61K 2800/884

(57) ABSTRACT

Technologies are described for methods and compounds for mitigating dag on hair. The method comprises applying a monofunctional compound comprising a hydrophobic tail and a reactive head to the hair and applying a difunctional compound to the hair. The difunctional compound has a first functional component and a second functional component. The application of the difunctional compound enables the first functional component to react with the reactive head of the monofunctional compound, the second functional component to form a matrix in situ with the hair, and the hydrophobic tail of the monofunctional compound to extend from the hair. The hydrophobic tail imparts its hydrophobicity to the hair to mitigate the dag.

### CROSS REFERENCE TO RELATED APPLICATIONS

This application is related to contemporaneously filed U.S. patent application Ser. No. 15/273,143, filed Sep. 22, 2016, titled “Methods and Compositions for Dag Mitigation”, by Georgius Adam, having attorney docket number JL1120.038, herein incorporated by reference.

### TECHNICAL FIELD

The present disclosure is directed generally towards methods and compositions for mitigating dag, and more specifically towards mitigating dag on the hair of animals, especially livestock.

### BACKGROUND

Dag often forms with water, urine, and defecation. The dag may also grow over time. The formation of dag may lead to an enhanced probability of disease as large amounts of bacteria may be introduced into the dag. Dag formation on cattle, and problems associated therewith, may be prevalent in many places. For example, cattle are often exported in large ships. The dags may add considerable weight and should be removed from the cattle before boarding to prevent excess shipping weight, disease, and for passing inspection.

Dags adhered to the hair of livestock may also represent a significant health problem for the beef processing industry. This may be especially true during seasonal periods of rain. Processing of “daggy” cattle at abattoirs may increase the risk that meat reaching the consumer is contaminated with pathogenic microorganisms.

Current methods for dag removal may induce stress in live animals at feedlots. This may have a detrimental effect on the quality of meat reaching the consumer, and additionally may pose health and safety hazards for workers. While cleaning and removal of the dags may be performed at the abattoir before or following slaughter, high microbial loads may be present in dags which may threaten food safety protocols.

Unless otherwise indicated herein, the materials described in this section are not prior art to the claims in this application and are not admitted to be prior art by inclusion in this section.

### SUMMARY

Technologies are generally described for methods and compounds for mitigating dag on hair. Mitigating dag may ease the removal of dag on hair, or may reduce, or even eliminate, the need to remove the dag. For example, mitigating the formation of dag may reduce the need to remove dag.

Methods and compositions for mitigating dag on hair are presently disclosed herein. A method comprises applying a monofunctional compound comprising a hydrophobic tail and a reactive head to hair and applying a difunctional compound to the hair. The difunctional compound has a first functional component and a second functional component. The applying of the difunctional compound enables the first functional component to react with the reactive head of the monofunctional compound, the second functional component to form a matrix in situ with the hair, and the hydrophobic tail of the monofunctional compound to extend from the hair. The hydrophobic tail imparts its hydrophobicity to the hair and mitigates the dag.

Another method of mitigating dag on hair comprises applying a compound having at least one hydrophobic component and at least two reactive functional components to the hair. A binder is also applied to the hair. The binder is selected from the group consisting of a curing agent, a catalyst, a hardener, a crosslinking agent, and combinations thereof. The applying of the compound and the binder to the hair enables a reaction with the hair and the formation of a matrix in situ with the hair, the matrix has the at least one hydrophobic component extending from the hair and imparts its hydrophobicity to the hair to mitigate the dag.

The foregoing summary is illustrative only and is not intended to be in any way limiting. In addition to the illustrative aspects, embodiments, and features described above, further aspects, embodiments, and features will become apparent by reference to the examples and drawings and the following detailed description.

### BRIEF DESCRIPTION OF THE FIGURES

The foregoing and other features of this disclosure will become more fully apparent from the following description and appended claims, taken in conjunction with the accompanying drawings and examples. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates a monofunctional compound having a hydrophobic tail and a reactive head;

FIG. 2A illustrates a difunctional compound having a first functional component reacted with a head of a monofunctional compound and a second functional component forming a matrix with a substrate;

FIG. 2B illustrates an exploded portion of the matrix shown in FIG. 2A;

FIG. 3 illustrates a reaction scheme of the present disclosure;

FIG. 4 illustrates the hydrophobicity of hair treated by the method of the present disclosure;

FIG. 5 illustrates the hydrophobicity of a substrate treated by the method of the present disclosure;

FIG. 6 illustrates a method of mitigating dag on hair of the present disclosure.

FIG. 7 illustrates a reaction scheme of the present disclosure;

FIG. 8 illustrates a reaction scheme of the present disclosure;

FIG. 9 illustrates a reaction scheme of the present disclosure;

FIG. 10 illustrates a reaction scheme of the present disclosure; and

FIG. 11 illustrates a reaction scheme of the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Dag, daglock, or daggle-lock is a lumpy, dirty, or clotted hair mass that has accumulated on the hair of livestock, such as cattle or sheep, and other animals. For example, dag may be a dangling or matted lock of fur, hair, or wool and may comprise feces or urine. For this disclosure, a dag is considered to be any foreign matter that clings to the hair, wool or other mammalian hair of an animal.

The presently disclosed method may not involve activities and factors that are known to be stressful to cattle such as noisy environment, human handling, electric prodding, washing, dipping, brushing, and shearing. For example, the compounds of the present disclosure may be passively applied to the hair of livestock.

The selective compounds and components disclosed may protect the hair of cattle from wetting. Since dags may need wet hair to form, the mitigation of wetting of the hair may then in turn mitigate the formation of dags on the hair. This may be especially true during the rainy or winter months. The presently disclosed method and compounds may protect or inhibit wetting of the hair for several months. Additionally, the presently disclosed method may ease the removal of dags that may form on the hair.

In an illustrative example of the present disclosure, a method of using a monofunctional hydrophobic compound(s) and Bifunctional compound(s) that are reactive with each other and are reactive, or enabled to become reactive, with hair, for example keratin or cysteine, is disclosed. The reaction may transform the hair to a protective substantially non-wetable hydrophobic composition that eases the removal, or mitigates the accumulation, of dags on hair. Dags may have a composition that is mostly hydrophilic, due to the presence of partially digested cellulose and sugar residues, which may also contribute to the mitigation of dag on hair, treated by the presently disclosed method.

In another illustrative example, a method of mitigating dag on hair comprises applying a compound having at least one hydrophobic component and at least two reactive functional components to the hair and applying a binder to the hair. The binder is selected from the group consisting of a curing agent, a catalyst, a hardener, a crosslinking agent, and combinations thereof. The applying of the compound and the binder to the hair enables a reaction with the hair and the formation of a matrix in situ with the hair. The matrix has the at least one hydrophobic component extending from the hair and imparts its hydrophobicity to the hair to mitigate the dag.

In at least one illustrative example of the present disclosure, a method of transforming the hair to be hydrophobic by reacting hydrophobic, or super hydrophobic, reactive hydrocarbon based active agent(s), or compound(s), with keratin and/or cysteine, the essential component of hair, is provided. Keratin is a protein formed by the combination of 18 amino acids, among which cysteine being rich in sulfur plus other reactive functional groups that may play an important role in the cohesion of hydrophobic compounds to the hair. Cysteine represents the major active component of keratin, which may be used for changing the properties of hair from hydrophilic to hydrophobic. Coloring pigments in hair, such as melanin, may also have reactive functional groups which may also be effective in transforming hair from hydrophilic to hydrophobic. For example, a difunctional compound may react with keratin or cysteine in the hair and hold the hydrophobic tail of a monofunctional compound to the hair.

This disclosure is generally drawn, inter alia, to methods and compounds for mitigating dag on hair. Briefly stated, technologies are generally described for a method and compounds for easing removal of dag from hair or reducing, mitigating, or substantially eliminating the formation of dag on hair.

Disclosed herein is a method of mitigating dag formation on hair. The method involves applying, for example spraying, compounds onto the hair. One or more of the compounds may be hydrophobic and may function as a water proofing agent or water repellant. Monofunctional compounds of the present disclosure applied to the hair may have a hydrophobic hydrocarbon containing component of hydrophobic tail and a reactive component or reactive head. Difunctional or polyfunctional compounds of the present disclosure may have a first functional component and a second functional component. The polyfunctional compound may also have a third, or more, functional component. These functional components may function similarly to the first and/or second functional components as disclosed herein. For example, the polyfunctional compound may have two or more functional components that may bind with hair or may have two or more functional components that may bind with the reactive head of the monofunctional compound. The application of the compounds may enable an in situ reaction of the compounds with the hair. The reaction may cause the compounds to form a matrix with the hair and impart the hydrophobicity of the tail of the monofunctional component to the hair and thereby mitigate the dag.

In an illustrative example, a hydrophobic hydrocarbon containing compound, or monofunctional compound comprising a hydrophobic tail and a reactive head, and a reactive difunctional compound may be applied to the hair simultaneously or separately. For example, the monofunctional compound and the difunctional compound may be mixed together and applied to the hair in a single application. In at least one illustrative example, the monofunctional compound and the difunctional compound are parts of a single compound that may be applied in a single application. In another illustrative example, one of the monofunctional compound and the difunctional compound are applied to the hair and then the other of the monofunctional compound and the difunctional compound is applied, in separate applications.

The monofunctional and difunctional compounds may be applied simultaneously in a single application or in separate applications. For example, the monofunctional and difunctional may be mixed together and immediately applied. Alternatively, the monofunctional and difunctional compounds may be applied separately and at the same time.

Regardless whether the monofunctional and difunctional compounds are applied as a mixture or separately, upon the application of both compounds to the hair, they may react with the hair and bond or hold the hydrophobic tail of the monofunctional compound to the hair. The hydrophobicity of the hydrophobic tail of the monofunctional compound may thus be transferred to the hair. More specifically, the applied reactive compounds may enable a reaction with the hair that binds the applied hydrophobic hydrocarbon, hydrophobic tail, to the hair and enable a transfer of its hydrophobicity to the hair to mitigate dag.

In another illustrative example, a method of mitigating dag on hair comprises applying a compound having at least one hydrophobic component and at least two reactive functional components to the hair and applying a binder to the hair. The binder is selected from the group consisting of a curing agent, a catalyst, a hardener, a crosslinking agent, and combinations thereof. The applying of the compound and the binder to the hair enables a reaction with the hair and the formation of a matrix in situ with the hair. The matrix has the at least one hydrophobic component extending from the hair and imparts its hydrophobicity to the hair to mitigate the dag.

One or more compounds may be in liquid form and the step(s) of applying the compounds to the hair may include spraying, rinsing, dispersing, or applying by means which are known by persons having ordinary skill in the art for applying a liquid to hair. One or more compounds may be in solid or powder form and the application of the compounds to the hair may include dusting, sprinkling, or applying by means which are known by persons having ordinary skill in the art for applying solids to hair.

Additional materials may also be applied to the hair. For example, one or more hardeners, catalysts, solvents reactive diluents, bonding agents, crosslinking agents, or other materials may be applied to the hair which may enhance, speed up, or drive a bonding reaction which may hold the hydrophobic tail of the monofunctional compound, or the compound having at least one hydrophobic component and at least two reactive functional components, to the hair.

In describing more fully this disclosure, reference is made to the accompanying drawings and following examples in which illustrative embodiments of the present disclosure are shown. This disclosure may, however, be embodied in a variety of different forms and should not be construed as so limited.

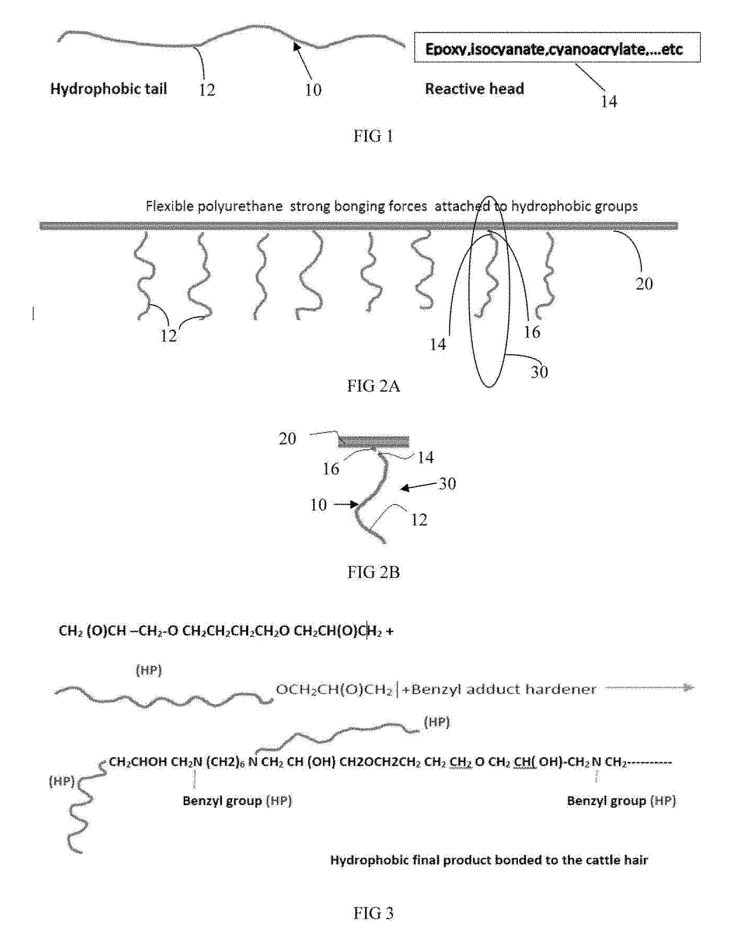

FIG. 1 illustrates a monofunctional compound 10 having a hydrophobic tail 12 and a reactive head 14. The hydrophobic tail may comprise hydrocarbon chain(s) or ring(s) and the reactive head may be reactive with the difunctional compound of the present disclosure. For example, reactive head 14 may comprise epoxy, isocyanate, cyanoacrylate, or other reactive components as are known in the art.

FIG. 2A illustrates a difunctional compound 16 having a first functional component reacted with reactive head 14 of monofunctional compound 10 and a second functional component forming a matrix with a substrate 20. FIG. 2B illustrates an exploded portion 30 of the matrix shown in FIG. 2A. As illustrated, the application of difunctional compound 16 and monofunctional compound 10 (shown in FIG. 1) to substrate 20 enables a first functional component (i.e. the end of difunctional compound 16 reacted with monofunctional compound 10), of difunctional compound 16, to react with reactive head 14 of monofunctional compound 10, and a second functional component (i.e. the end of difunctional compound 16 reacted with substrate 20), of difunctional compound 16, to form a matrix in situ with substrate 20. The matrix has a plurality of hydrophobic tails 12, of the monofunctional compounds 10, extending from substrate 20 and imparting their hydrophobicity to substrate 20. In this illustrative example, substrate 20 may illustrate hair and the imparting of the hydrophobicity to the hair may mitigate dag on the hair.

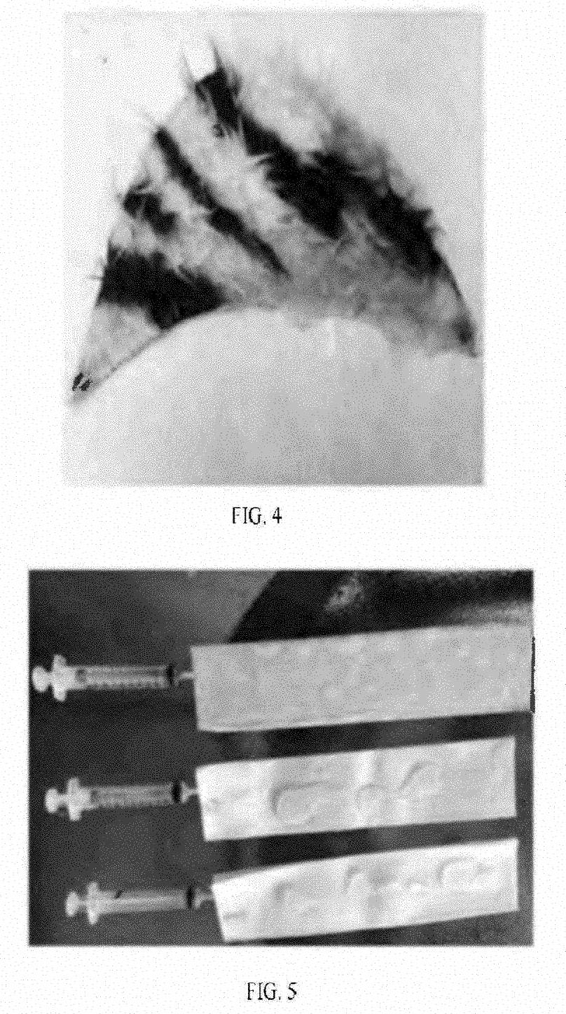

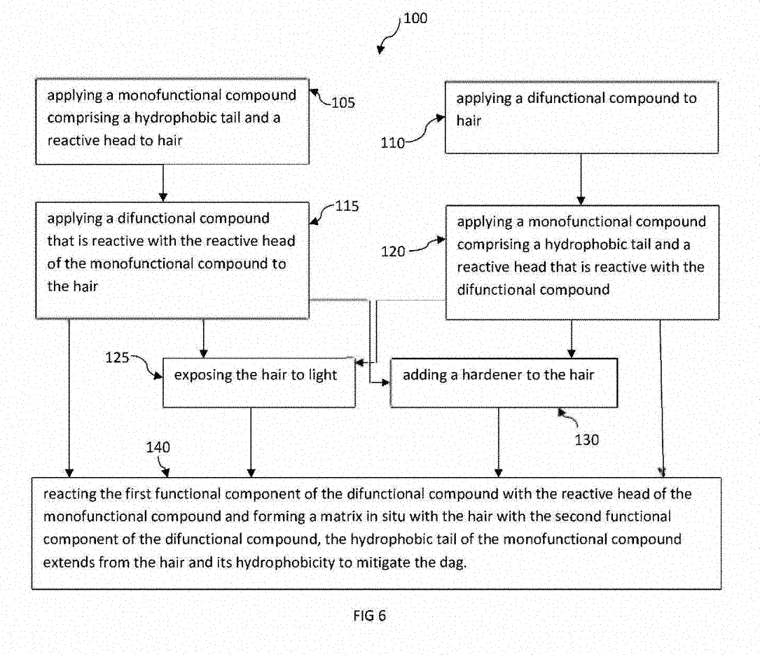

FIG. 3 illustrates a reaction scheme that may take place upon applying the monofunctional and difunctional compounds to hair to form a matrix. The reactions with the monofunctional compound, difunctional compound, and hair may be mild and may take place in situ, at ambient conditions. The mechanism of the reactions that takes place may be based on changing the structure of cysteine and hydrogen bonding groups of cysteine, COOH, NH₂, NH, C═O, for bonding of the hydrophobic tail. Several types of these reactive ingredients may be available commercially as reactive starting material.

A catalyst may also be applied to the hair. The concentration of the catalyst required to control the rate of curing of the final hydrocarbon may be between about 0.01-0.001 weight % of the total composition. The catalyst may comprise a composition selected from the group consisting of stanous octanoate, dialkyltin oxide, tetra-n-propyl orthosilicate, dioctyltin oxide, platinum complex catalysts, dibutyltin dilaurate, dibutyltin dioctanoate, and combinations thereof. The catalyst composition may have an inert solvent to dilute the catalyst as the concentration of the catalyst used may be very low compared to the total composition. The provided catalyst may be added to a container and mixed into the composition comprising a monofunctional component and/or difunctional component. The composition and provided catalyst may be simultaneously and immediately applied to the hair, immediately upon adding the provided catalyst to the container and mixing. Alternatively, the provided catalyst may be added to the solution, or applied to the hair, after the application of the monofunctional or difunctional compound is applied onto the hair.

A photocatalyst may also be applied onto the hair. The photocatalyst may be applied simultaneously with the monofunctional and/or difunctional compounds. Upon exposing the hair to light, a photocatalytic reaction of the monofunctional and difunctional compounds with the hair may take place.

A composition containing a hardener may also be applied to the hair. The hardener may be optional. For example, a hardener may be applied to hair as a composition at an equivalent ratio to the monofunctional and difunctional compounds for curing and bonding and forming a matrix with the hair. The hardener may comprise an amine terminated polypropyleneoxide, an amine terminated polybutadiene, isophoron diamine, meta xylene diamine, or other hardener as is known by persons having ordinary skill in the art for forming a matrix of the monofunctional and difunctional compounds with the hair.

The hardener and the monofunctional and/or difunctional compounds may be simultaneously applied to hair by mixing the hardener with the monofunctional and/or difunctional compounds and immediately applying to the hair. In at least one embodiment of the present disclosure, the monofunctional and/or difunctional compounds and a hardener are applied separately to the hair, in at least two separate applications. For example, a monofunctional and/or difunctional compound may be sprayed, or otherwise applied, onto hair and separately, a hardener may be sprayed, or otherwise applied, onto the hair.

FIG. 4 illustrates an untreated surface of an animal on the right side and an underlying hydrophobic matrix on the left side. The right side portion depicts the untreated surface of the animal. Shown on the left side is a portion of the hair that has had the monofunctional and difunctional compounds of the present disclosure applied to the hair. As depicted in the left side and explained below, the applied reactive compounds enables a reaction with the hair that binds the applied hydrophobic hydrocarbon containing compound, or monofunctional compound, to the hair and the applied difunctional compound enables binding of the monofunctional compound and a transfer of its hydrophobicity to the hair to mitigate dag on the hair. For example, the hair may be livestock hair and the hydrophobic compounds may have their hydrophobic tails extending from the hair. The left side of the hair was treated according to the method of the present disclosure and water was applied to the hair. As clearly shown in FIG. 4, the treated left side is substantially more hydrophobic than the untreated right side which may mitigate dag on hair.

FIG. 5 illustrates the hydrophobicity of a substrate treated by the method of the present disclosure by illustrating the water repellency or hydrophobic characteristics of applied compounds. The first illustration in FIG. 5, the picture shown at the top, shows untreated tissue paper that has been sprayed with water. As shown in the first illustration, the untreated tissue paper is hydrophilic and has absorbed the water. The second and third illustrations in FIG. 5, depicted as the middle and bottom pictures, show tissue papers that have been treated by the method of the present disclosure and then sprayed with water. More specifically, the tissue paper in the second and third illustrations were treated by spraying them with monofunctional and difunctional compounds of the present disclosure wherein the difunctional compound enabled its first functional component to react with a reactive head of the monofunctional compound. A second functional component of the difunctional compound reacted with the tissue paper and formed a matrix in situ therewith. The hydrophobic tail of the monofunctional compound extended from the tissue paper and imparted its hydrophobicity thereto.

As shown in the second and third illustrations, the treated tissue paper is made hydrophobic due to the applied compounds of this disclosure and has repelled the sprayed on water to the extent that the water has pooled on its surface. A comparison of the first with the second and third illustrations of FIG. 5 clearly shows that the application of the monofunctional and difunctional compounds according to the present disclosure enables a reaction with the substrate and transfers the hydrophobicity of the hydrophobic tail of the monofunctional compound to the tissue paper.

FIG. 6 illustrates the method of mitigating dag on hair of the present disclosure. A method of mitigating dag on hair 100 comprises applying a monofunctional compound comprising a hydrophobic tail and a reactive head to the hair at step 105. At step 115 a difunctional compound is applied to the hair. Alternatively, a difunctional compound may be applied to the hair at step 110 and followed by the application of the monofunctional compound comprising a hydrophobic tail and a reactive head to the hair at step 120. In another illustrative example, the monofunctional and difunctional compounds may be applied simultaneously. The hair may be exposed to light at step 125 and/or a hardener may be applied to the hair at step 130. Steps 125 and 130 are optional as the monofunctional and difunctional compounds may react with the hair to form a matrix without these steps being performed. It is to be understood that other or additional steps may be performed or additional materials may be applied to the hair to provide a desired hydrophobicity to the hair. Upon applying the monofunctional and difunctional compounds to the hair and performing any additional desired steps or applying additional materials, the difunctional compound enables its first functional component to react with the reactive head of the monofunctional compound, its second functional component to form a matrix in situ with the hair, and the hydrophobic tail of the monofunctional compound to extend from the hair, the hydrophobic tail imparts its hydrophobicity to mitigate the dag at step 140.

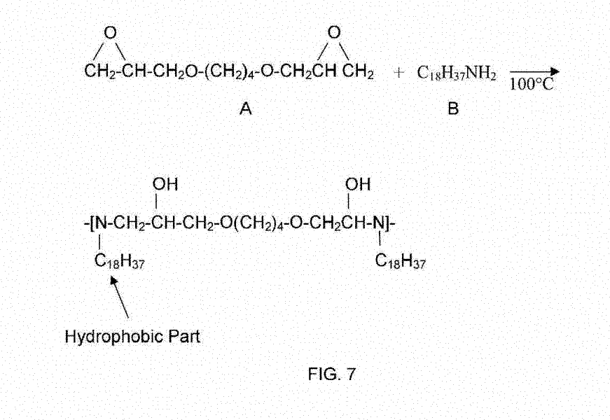

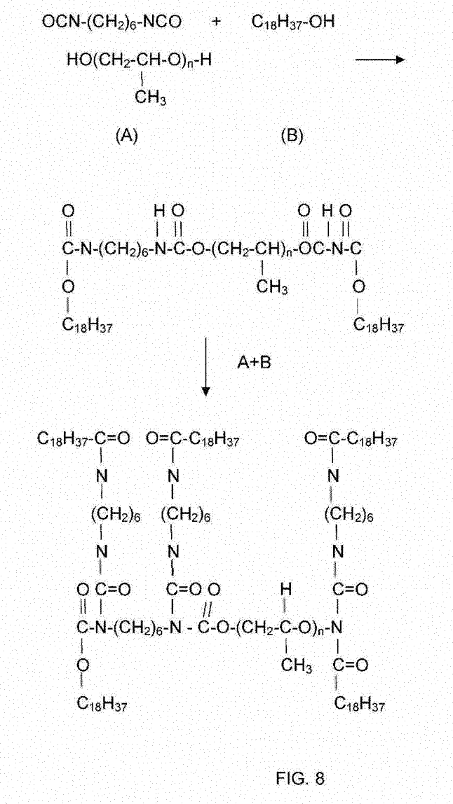

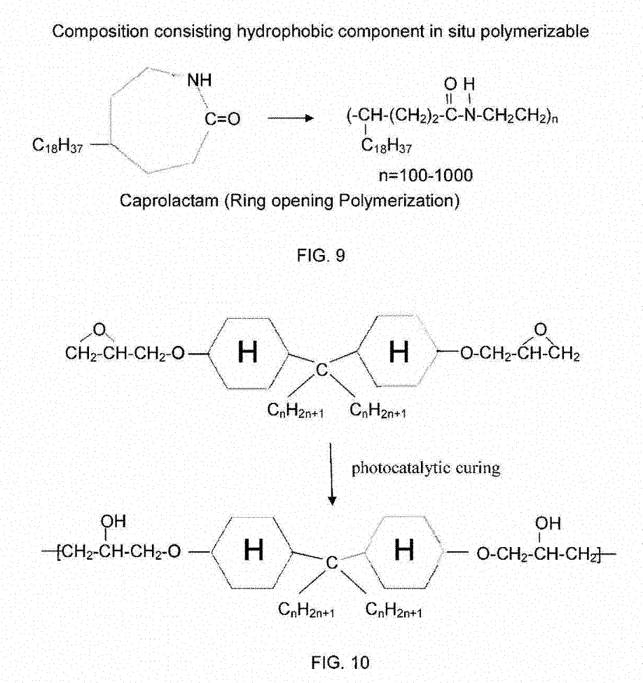

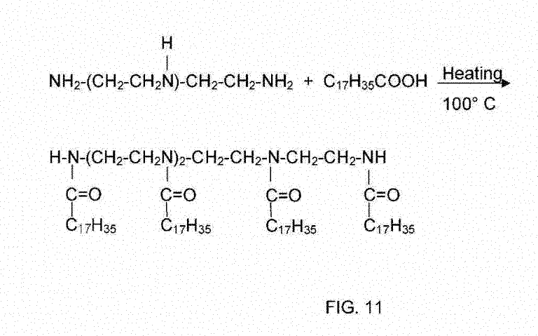

FIGS. 7 through 11 illustrate reaction schemes for a method of mitigating dag on hair comprising applying a compound having at least one hydrophobic component and at least two reactive functional components to the hair and applying a binder to the hair. The binder is selected from the group consisting of a curing agent, a catalyst, a hardener, a crosslinking agent, and combinations thereof. The applying of the compound and the binder to the hair enables a reaction with the hair and the formation of a matrix in situ with the hair, the matrix has the at least one hydrophobic component extending from the hair and imparts its hydrophobicity to the hair to mitigate the dag.

In an illustrative method, mitigating dag on hair comprises applying a monofunctional compound comprising a hydrophobic tail and a reactive head to hair and applying a difunctional compound to the hair. The monofunctional compound and the difunctional compound may be applied separately or mixed together and applied simultaneously. The difunctional compound has a first functional component and a second functional component. The first functional component is reactive with a reactive head of the monofunctional compound. The application of the difunctional compound enables the first functional component to react with the reactive head of the monofunctional compound, the second functional component to form a matrix in situ with the hair, and a hydrophobic tail of the monofunctional compound to extend from the hair. The matrix formed with the hair imparts the hydrophobicity of the tail of the monofunctional compound to the hair and mitigates the dag.

The term hair is used broadly herein to mean any form of mammalian hair such as wool or any of the fine threadlike strands growing from the skin. The term hide is used broadly herein to mean the skin from which the hair is growing. For example, the method and compounds of the present disclosure may ease removal of dag from hair and/or reduce, or even eliminate, the formation of dag and a need to remove dag from the hair of cattle. It will be appreciated that by reducing or even eliminating dag on the hair of cattle, this disclosure helps keep dag away from the hide of the cattle or ease the removal of formed dags.

The current disclosure may provide a non-stress-inducing method of mitigating dags on hair. The mitigation of dags may mitigate the contamination of the meat. The reduction, mitigation, or elimination of the dags may, in turn, aid the slaughter house in meeting regulations that may require that the cattle be deemed “clean” prior to slaughter. In addition, post-slaughter de-dagging methods may not be a viable option. This may be especially true in meeting Australian regulations.

The invention is based on using hydrocarbon based hydrophobic compounds, reactive hydrocarbon based hydrophobic agents, and/or water repellent ingredients that may become reactive with keratin, melanin, or other hair components. A reaction of the compounds with hair, for example cattle hair, may cause a matrix to form in situ with the hair and a hydrophobic compound to become chemically bonded with hair. The hydrophobic compound may became part of the cattle hair and keep the cattle substantially dry and clean. The method may be tailored to be persistent in keeping the hair substantially dry for several months or until the hair of the cattle is renewed or the cattle is slaughtered. The reaction of the compounds with the hair may be a mild substantially non-exothermic reaction forming matrix with the hair.

The presently disclosed method of mitigating dag on hair may be based on the chemistry of surfactants and the hydrophobic materials. The chemical structure of the surfactants may be anionic, cationic, non-ionic, or molecular. The surfactant may have a hydrophobic tail which may have side chain hydrocarbons, such as aliphatic, aromatic, mixed, linear or branched hydrocarbons. Other types of surfactants, such as Gemini surfactants, may have a plurality of hydrophobic tails which may provide advantages in mitigating dag over the anionic, cationic, non-ionic, and molecular surfactants.

In an illustrative example, the presently disclosed method of mitigating dag on hair may be based on using cost effective organic hydrophobic agents or compounds that may react with compositions of epoxy resins, polyurethanes, or poly cyanoacrylate, at ambient temperatures, and form strong bonding with keratin, cysteine, or melanin components of hair to form a matrix with the hair. A matrix formed on cattle hair, for example, may comprise hydrophobic agents bonded chemically to the hair which will became part of the cattle hair to keep the cattle substantially dry and clean. The compounds forming the matrix may be tailored to be persistent for several months or until the hair of the cattle is renewed.

Direct application or spraying of organic hydrophobic agents and surfactants on cattle to prevent dag accumulation may not be successful because they may not bind with the hair and may be washable and non-stable. The disclosure is based on transferring the hydrophilic head of the surfactants to reactive groups that may form chemical bonds with hair constituents such as epoxy, cyanoacrylates, isocyanates, amine, alcohol and others. A method of mitigating dag on hair of the present disclosure may comprise applying a monofunctional compound comprising a hydrophobic tail and a reactive head to hair and applying a difunctional compound to the hair, the difunctional compound having a first functional component and a second functional component. The application of the monofunctional and difunctional compounds may enable the formation of a matrix in situ with the hair and impart the hydrophobicity of the tail to the hair and mitigate the dag.

The monofunctional compound may be made by substituting, or bonding to, a hydrophilic head of a surfactant with the difunctional compound. For example, hydrocarbon based surfactants, such as conventional surfactants and Gemini surfactants, may have a hydrophilic head that is reactive with a Bifunctional compound of the present disclosure. For example, surfactants that may be used make the monofunctional compound of the present disclosure may be illustrated by the following structures.

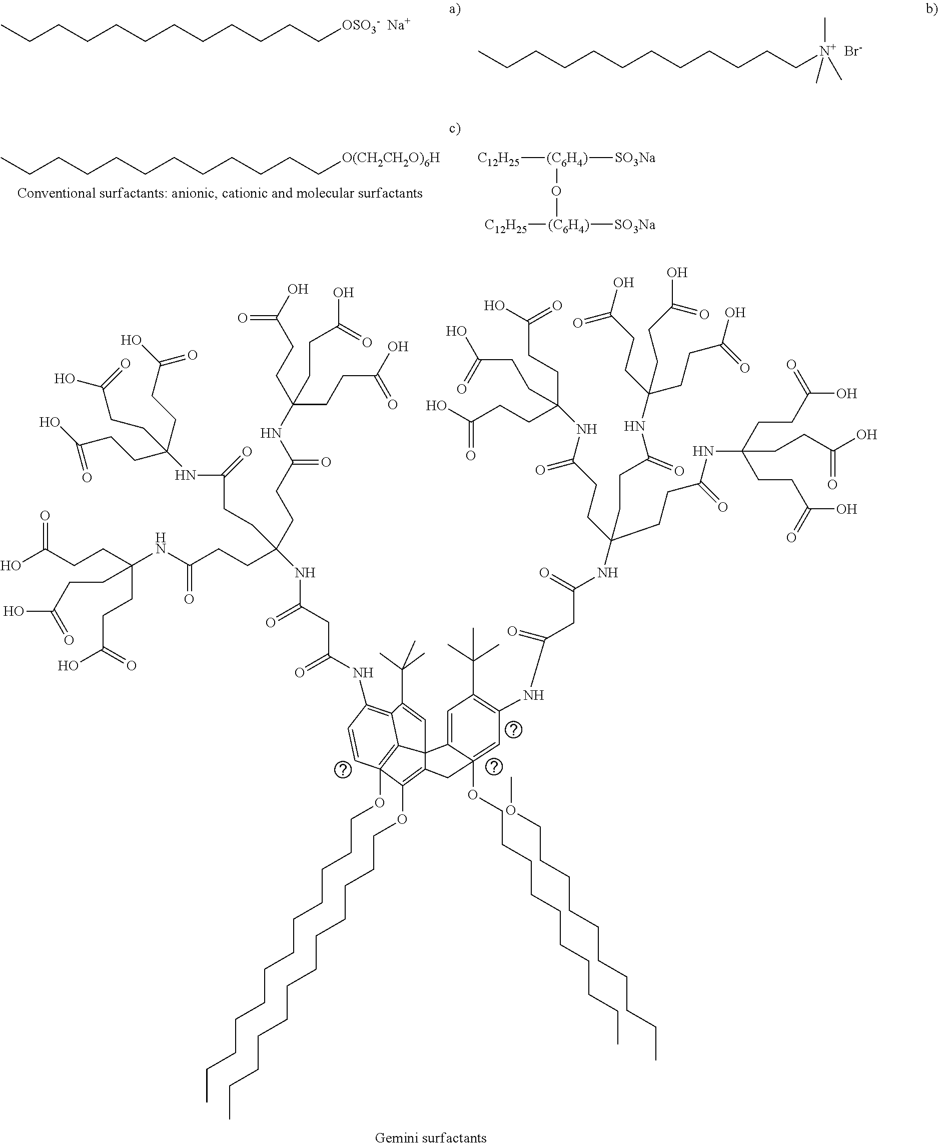

Other constituents that comprise hydrophobic tails that may be used in the presently disclosed method of mitigating dag may be constituents that are currently used in water preservation. For example, constituents used to prevent or reduce the rate of water evaporation from dams, swimming pools, and other water reservoirs, may have hydrophobic tails that may be used to make the monofunctional composition of the present disclosure. In an illustrative example, long chain fatty alcohols such cetyl alcohol that are sprayed on the surface of water reservoirs to prevent or retard water evaporation may be used as a source of a hydrophobic tail. The constituents used to prevent or reduce the rate of water evaporation may have a hydroxyl alcoholic group which may form hydrogen bonding with water molecules on the surface and have a long chain hydrophobic hydrocarbon that forms hydrophobic layer on the surface prevents, or reduces, water from evaporation. The hydroxyl alcoholic group in these examples may provide a site with which the difunctional component of the present disclosure may bond.

For example, the monofunctional compound have one or more of:

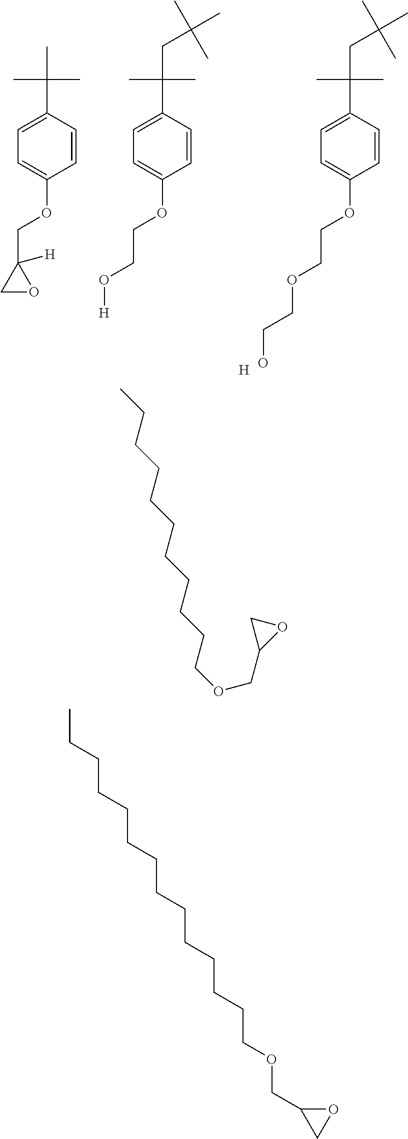

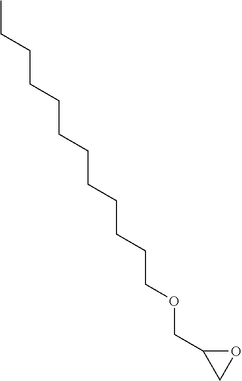 [SMILES: CCCCCCCCCCCCOCC1CO1]

a Gemini surfactant, a flexible long hydrocarbon chain having between 6 and 20 carbon atoms,

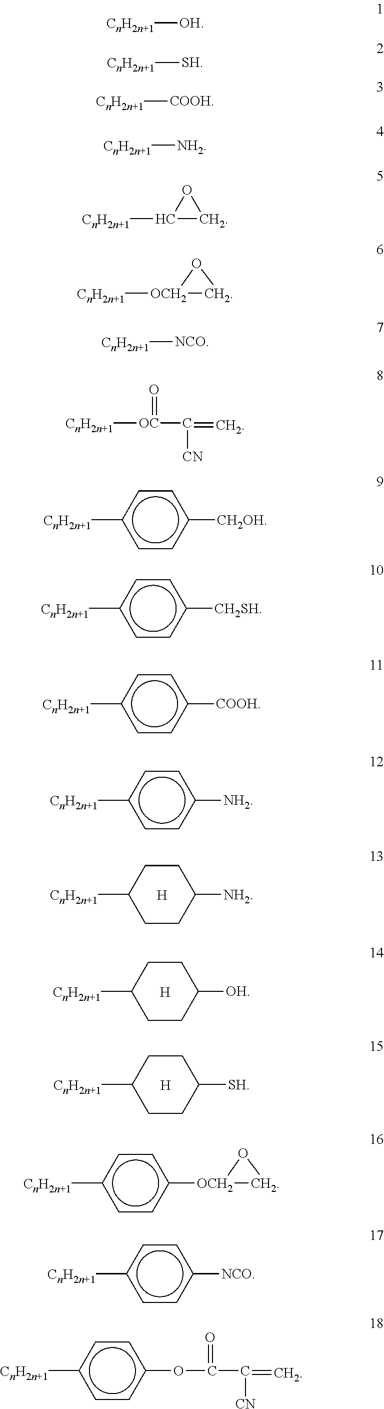

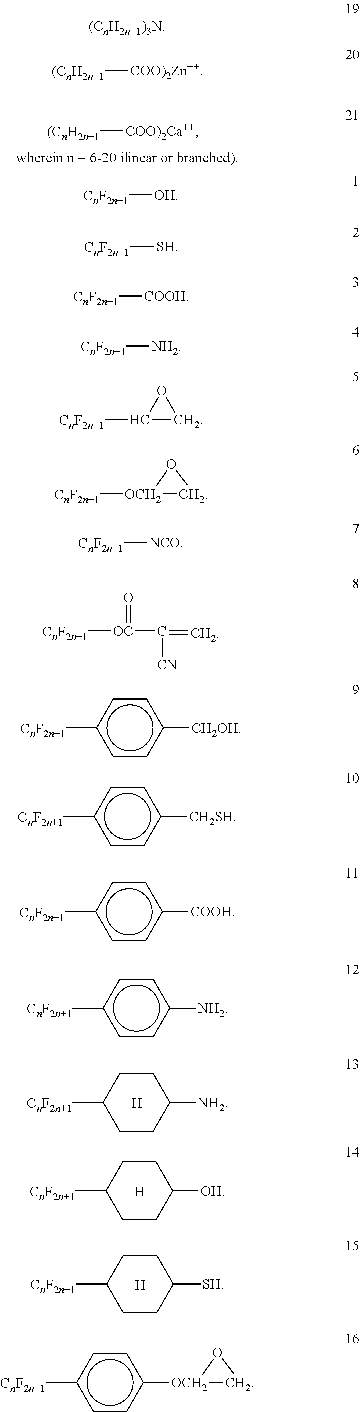

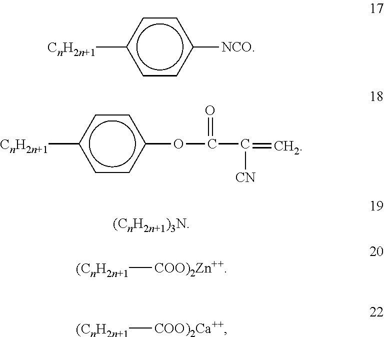

wherein n=6-20 (linear or branched)
Reactive composition that contains at least one hydrophobic group and at least one reactive polymerisable group that adhere to the hair components:

1. Cyanoacrylates

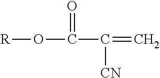

R, R₁, R₂=C₆-C₂₀ linear, branched, cydoaliphatic, alkyl phenyl, hydrocarbon chain, aliphatic, aromatic, cycloaliphatic, alkyl phenyl, alkyl cycloaliphatic

2. Glycidyl Ethers

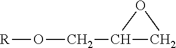

3. Caprolactam Derivatives

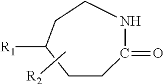

4. Caprolactone Derivatives

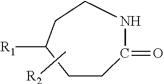

5. Polyurea

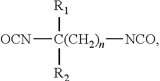

wherein n=2-8

6.

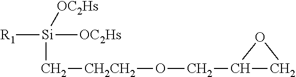

7

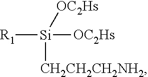

stearate hydrophobic chain, linear chain aliphatic glycidyl ether C₁₂-C₁₄ OCH₂CH(O)CH₂, C₁₀ glydicyl ether, iso octyl glycidyl ether, linear chain aliphatic glycidyl ether from C₈-C₁₀ OCH₂CH(O)CH₂, oleyl amine; C₁₉H₃₈ NH₂, nonylphenylmonoglycidylether; C₉H₁₉ C₆H₄ O CH₂ CH(O)CH₂, t-butylphenylmonoglycidylether, C₄H₉C₆H₄O CH₂CH(O)CH₂, penta fluoro benzylalcohol C₇F₅H₂OH, and metaxylene diamine.

The difunctional compound may comprise one or more of: 1,4 butandiol di glycidylether(1,1,1-triglycidylethertrimethylolpropane, neopentyldiglycidylether, stearic acid, oleyl amine, isophorone diamine, pentaethylene triamine, polypropylene diamine, and bisphenol-A diglycidylether containing o-cresylmonoglycidyl ether In at least one illustrative example of the presently disclosed method of mitigating dag, the monofunctional compound is C₁₃-monoglycidy ether and the difunctional compound is butanediol di glycidyl ether.

Presently disclosed is a method of mitigating dag on hair. A monofunctional compound comprising a hydrophobic tail and a reactive head is applied to hair. A difunctional compound having a first functional component and a second functional component is applied to the hair. The difunctional compound enables the first functional component to react with the reactive head of the monofunctional compound and the second functional component to form a matrix in situ with the hair. The matrix has the hydrophobic tail of the monofunctional compound extending from the hair, which imparts its hydrophobicity to the hair and mitigates the dag.

The monofunctional compound and the difunctional compound may be applied separately or may be mixed prior to applying to the hair. The reactive head or difunctional compound may have epoxy, acrylate, isocyanate, cyanoacrylate, amine, hydroxyl amine, alcohol, and combinations thereof.

The matrix formed with the hair may have one or more of monofunctional epoxy, isocyanate, alcohols, and amines as derivatives of fatty acids. For example, the matrix may comprise one or more of cetyl glycidyl ether, cresyl glycidyl ether, nonyl phenol glycidyl ether, n-butyl glycidyl ether, aliphatic glycidyl ether, 2-ethylhexyl glycidyl ether, phenyl glycidyl ether, o-cresyl glycidyl ether, p-tertiary butyl phenyl glycidyl ether, 3-alkyl phenol glycidyl ether, o-phenyl phenol glycidyl ether, and benzyl glycidyl ether.

In at least one illustrative example, a method of mitigating dag on hair comprises applying a hydrophobic material to the hair, wherein the hydrophobic material has a linking group and at least one of an aliphatic group, an aromatic group, and an aliphatic aromatic group with a chain consisting of C₆-C₂₀, and applying a binder that links with the linking group and binds with the hair. The application of the binder enables the hydrophobic material to form a matrix in situ with the hair and the hydrophobic material to extend from the hair, the hydrophobic material imparts its hydrophobicity to mitigate the dag on the hair.

The hydrophobic material may have one or more of stearate hydrophobic chain, linear chain aliphatic glycidyl ether from C₁₂-C₁₄ OCH₂CH(O)CH₂, linear chain aliphatic glycidyl ether such as: C₈-C₁₀ OCH₂CH(O)CH₂, oleyl amine C₁₉H₃₈, nonylphenylmonoglycidylether C₉H₁₉ C₆H₄OCH₂CH(O)CH₂, t-Butylphenylmonoglycidylether; C₄H₉C₆H₄O CH₂CH(O)CH₂, penta fluoro benzylalcohol C₇F₅H₂OH, and metaxylene diamine.

The binder may have one or more of 1,4 butandiol di glycidylether,1,1,1-triglycidylethertrimethylolpropane, neopentyldiglycidylether, stearic acid, oilyl amine, isophorone diamine, pentaethylene triamine, polypropylene diamine, and bisphenol-A diglycidylether containing 10% o-cresylmonoglycidyl ether epoxy equivalent 170 g/Eq wt.

In another illustrative example, a method of mitigating dag on hair comprises applying a compound having at least one hydrophobic component and at least two reactive functional components to the hair and applying a binder to the hair. The binder is selected from the group consisting of a curing agent, a catalyst, a hardener, a crosslinking agent, and combinations thereof. The applying of the compound and the binder to the hair enables a reaction with the hair and the formation of a matrix in situ with the hair. The matrix has the at least one hydrophobic component extending from the hair and imparts its hydrophobicity to the hair to mitigate the dag. The compound and binder prior may be mixed prior to applying to the hair. The formation of the matrix in situ with the hair may occur when the compound and the binder react at ambient temperature. The formation of the matrix in situ with the hair may occur when the compound and binder photocatalytically react.

The binder may be difunctional or polyfunctional. The binder may have at least one functional component selected from the group consisting of epoxy, acrylate, amine, isocyanate, hydroxyl, hydroxyl amine, alcohol, vinyl, allyl, cyanoacrylate, and combinations thereof. The binder may be selected from the group consisting of polyurethane, epoxy, polyurea, polyamide, polyester, polysiloxane, polyacrylate, and combinations thereof. The binder may comprise a photocatalytic catalyst and the photocatalytic catalyst may be applied to the hair in amount between about 0.01 and 0.1 weight percent of the compound and the binder.

The hydrophobic component of the compound having at least one hydrophobic component and at least two reactive functional components may have at least one hydrocarbon group with 6 to 20 carbon atoms. The compound may have at least one of the reactive functional components for reacting and bonding with the hair. The at least one functional component for reacting and bonding with the hair may be selected from the group consisting of epoxy, acrylate, amine, isocyanate, hydroxyl, hydroxyl amine, alcohol, vinyl, allyl, cyanoacrylate, and combinations thereof.

The compound may be selected from the group consisting of:

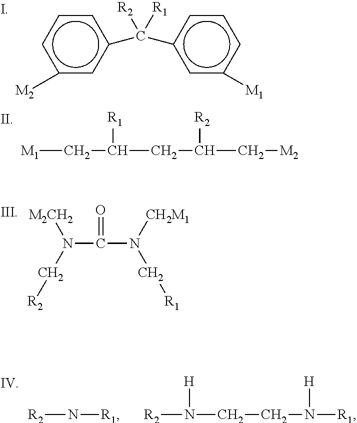

and combinations thereof. Wherein, M1 and M2 may be Glycidyl ether, acrylate, isocyanate, cyanoacrylate, amino, hydroxyl, vinyl, vinylether, allyl, allylether, or any other reactive functional groups. R1 and R2 may be Hydrocarbon groups with C8-C20, may be alkyl, alkylphenyl, cycloalkyl, phenyl, alkyl phenyl, the alkyl group may be linear or branched. All the R1 and R2 groups may be partially or fully fluorinated for production of super hydrophobic dag mitigating.

The compound having at least one hydrophobic component and at least two reactive functional components may be selected from the group consisting of:

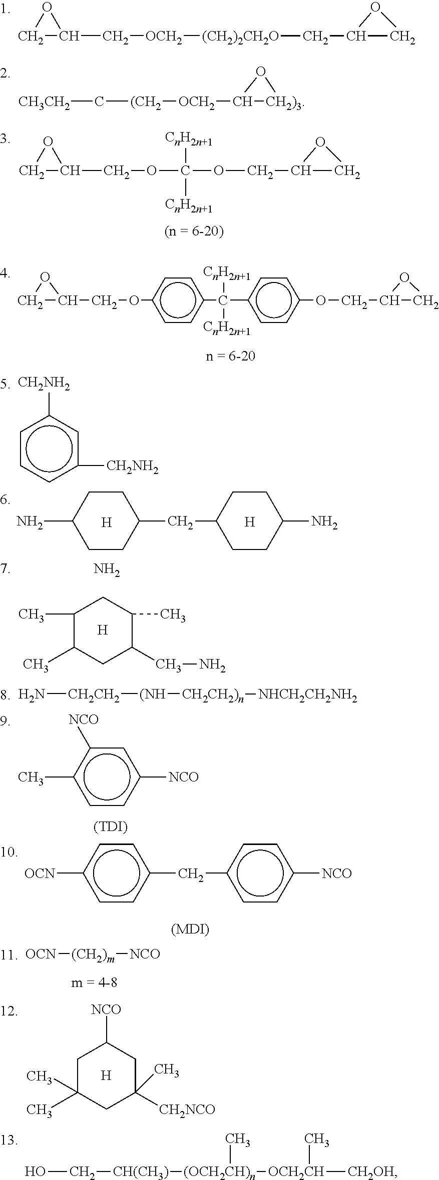

and combinations thereof.

The binder may comprise a catalyst or curing agent and the composition may be selected from the group consisting of chemical structures of compounds with at least one hydrophobic and at least two reactive functional components:

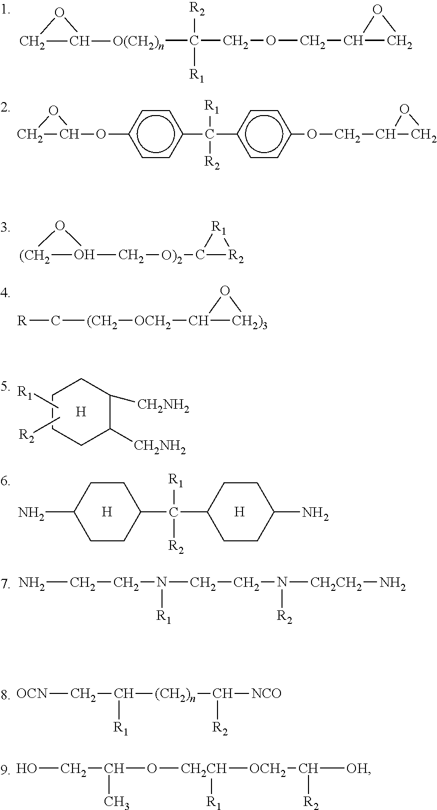

and combinations thereof.
R, R₁, R₂=C₆-C₂₀ (aliphatic, aromatic, cyclo aliphatic linear or branched)

The binder may be selected from the group consisting of chemical structures that act on binders for the hydrophobic and super hydrophobic ingredients and hair components:

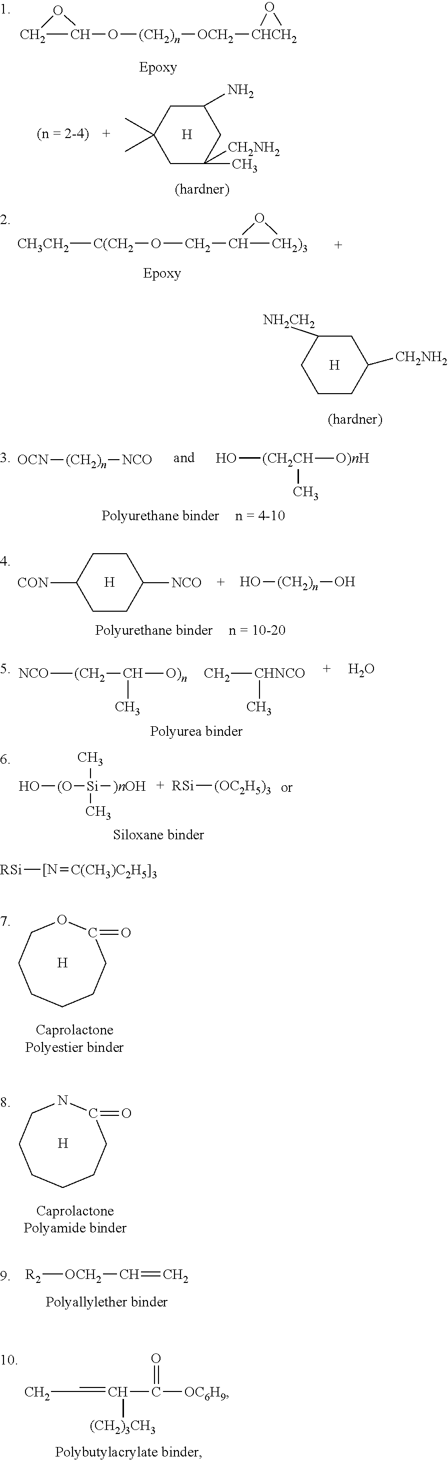

and combinations thereof.

The composition may comprise a reactive molecular Gemini surfactant, for example, a reactive molecular Gemini surfactant may be selected from the group consisting of:

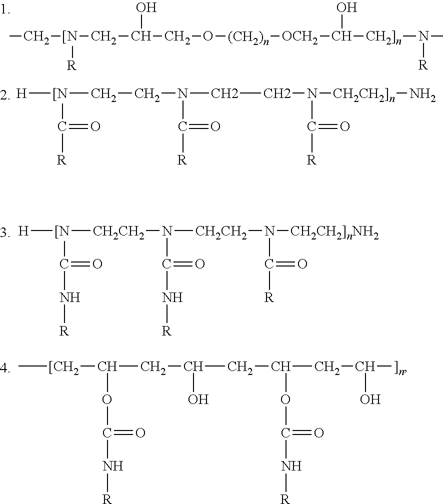

and combinations thereof.
n=50-100, R=C₆-C₂₀ [linear, branched, hydrocarbon chain, aliphatic, aromatic, acicyclic, alkylphenyl, chcloaliphatic, alkylcycloaliphatic. Structure of representative compounds that have Gemini structure Dag mitigating ingredients.

In at least one illustrative example, a method of mitigating dag on hair comprises charging a container with any of the compounds having at least one hydrophobic component and at least two reactive functional components or monofunctional compounds presently disclosed, for example stearyl amine. A presently disclosed binder or Bifunctional compound may be mixed into the container, for example 1,4-butanedioldiglycidylether and dodecylglycidylether may be mixed into the container and the dodecylglycidylether may function as a reactive hydrophobic component. A curing agent or hardener such as meta xylenediamine or tetraethylene pentamine may be mixed into the container to form a reactive composition. The reactive composition may be applied to the hair. Safe solvents or reactive diluents, such as alcohols, may be mixed into the container in an amount to make the reactive composition sprayable. The reactive composition may be cured on the hair for about 30 minutes to about 6 hours to form a matrix with the hair

In another illustrative example, a method of mitigating dag on hair comprises charging a container with tridecylglycidylether, trimethylolpropane triglycidylether, fluorinated benzylalcohol, tetraethylene pentamine and/or metaxylenediamine, and mixing the components into the container to form a reactive composition. The reactive composition may then be applied to the hair.

In a further illustrative example, a method of mitigating dag on hair comprises charging a container with stearyl amine or steric acid, mixing 1,4-butanediglycidylether into the container, mixing metaxylenediamine or tetraethylene pentamine into the container to form a mixture; and applying the mixture to the hair. Alcohol may be mixed into the container in an amount to make the mixture sprayable. The mixture may be cured on the hair for at least 30 minutes to form a matrix with the hair.

In yet another illustrative example, a method of mitigating dag on hair comprises charging a container with tridecylglycidylether or fluorinated benzylalcohol, mixing tetraethylene pentamine or metaxylenediamine into the container, mixing trimethylolpropane triglycidylether into the container to form a mixture; and applying the mixture to the hair.

### EXAMPLES

The following examples illustrate the method of mitigating dag on hair disclosed herein. The examples show selected applications of illustrative compounds according to the present disclosure. The contact angles observed in the examples are the angles where water meets the treated tissue paper, glass slides, hide, or leather. Typically, a contact angle smaller than 90° is considered hydrophilic and a contact angle larger than 90° is considered hydrophobic. As shown in the following examples, the observed contact angles exceeded 90°, indicating hydrophobicity of the matrix formed by the method and compositions of the present disclosure.

### Example 1

A 50 ml spray bottle was charged with 6.5 g of C13-monoglycidy ether (reactive hydrophobic agent, monofunctional compound) and 12.5 g of butanediol di glycidyl ether (difunctional compound) and 12 g of hardener composition (metaxylenediamine), mixed well and sprayed to the cow leather sample, shown in FIG. 4. The sprayed product cured rapidly at room temperature and formed strong bonds with the hair composition. The sample was then immersed in water, and evaluated. FIG. 4 shows the sample of cattle leather wherein the left part was treated with this reactive hydrophobic water repellent and the right part non-treated showing absorption of water.

### Example 2

A reaction vessel equipped with mechanical stirrer was charged with 500 g of 1,4 butandioldiglycidyl ether and 500 g of octadecylamine, heated at 100° C. for 6 hours under stream of nitrogen gas. The obtained solid epoxy had a melting point 70° C. and was dissolved in 10% ethanol and 20% reactive diluents such as triethoxy glycidylpropyl silane and amino ethyl aminopropyl tri ethoxy silane (1:1) and sprayed on paper tissue, cattle hide and glass. The measured contact angle was 143. This composition was applied to three cattle for final field evaluation.

### Example 3

The prepared solid epoxy in EXAMPLE 2 as 20% solution in ethanol was mixed with equivalent weight of 1,3 meta xylene diamine and sprayed on tissue paper, glass slides and hide. The reactive coating showed excellent stability when immersed in water for two weeks. The measured contact angle was 128.

### Example 4

A spraying bottle was charged with 12.4 g of 2,2-pentyl glycoldiglycidylether (equivalent weight 130), 23 g of nonylphenol glycidylether and 37 g of metaxylene diamine, mixed for 5 minutes and sprayed on tissue paper, glass slides and hide. The measured contact angle was 124. The cured reactive coating showed good water repellant and excellent stability when immersed in water for two weeks.

In at least one illustrative example, a monofunctional reactive hydrophobic agent(s), bifunctional reactive binder(s), and optional hardeners or curing agents are applied to hair to mitigate dag. For example, chemical structures that act on binders for the hydrophobic and super hydrophobic ingredients and hair components such as one or more of the following compounds may be applied to the hair.

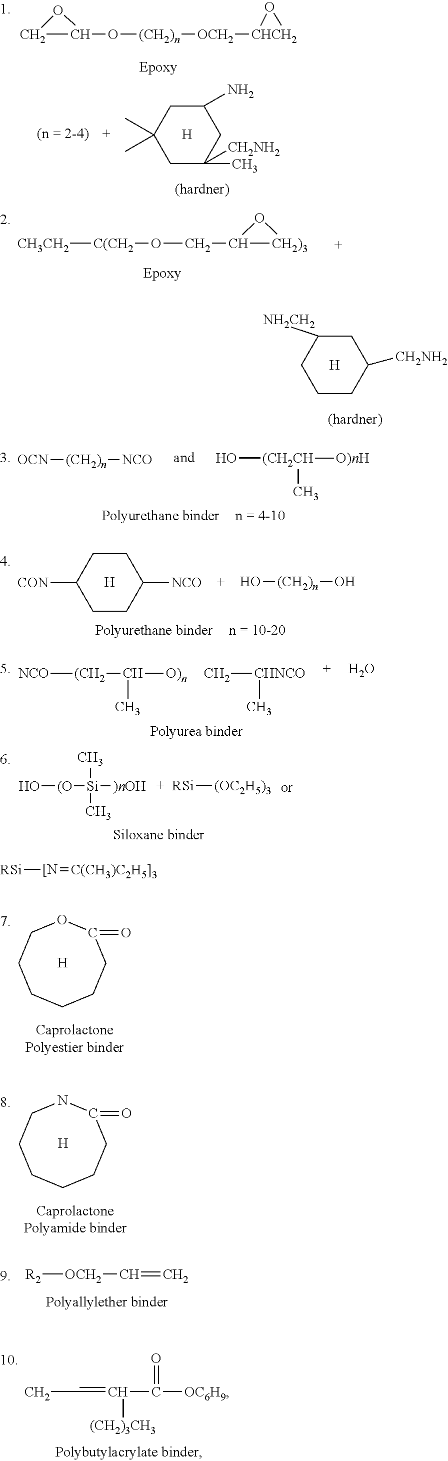

and combinations thereof.

In at least one additional illustrative example, one reactive composition that has both hydrophobic groups and reactive binding functional groups are applied to hair to mitigate dag. For example, reactive composition that contains at least one hydrophobic group and at least one reactive polymerisable group that adhere to the hair components, such as one or more of the following compounds may be applied to the hair.

1. Cyanoacrylates

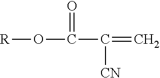

R, R₁, R₂=C₆-C₂₀ linear, branched, cydoaliphatic, alkyl phenyl, hydrocarbon chain, aliphatic, aromatic, cycloaliphatic, alkyl phenyl, alkyl cycloaliphatic

2. Glycidyl Ethers

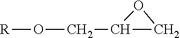

3. Caprolactam Derivatives

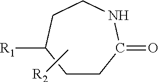

4. Caprolactone Derivatives

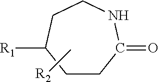

5. Polyurea

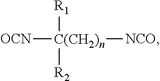

wherein n=2-8

6.

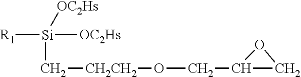

7.

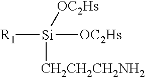

stearate hydrophobic chain, linear chain aliphatic glycidyl ether C₁₂-C₁₄ OCH₂CH(O)CH₂, C₁₀ glydicyl ether, iso octyl glycidyl ether, linear chain aliphatic glycidyl ether from C₈-C₁₀ OCH₂CH(O)CH₂, oleyl amine; C₁₉H₃₈ NH₂, nonylphenylmonoglycidylether; C₉H₁₉ C₆H₄ O CH₂ CH(O)CH₂, t-butylphenylmonoglycidylether, C₄H₉C₆H₄O CH₂CH(O)CH₂, penta fluoro benzylalcohol C₇F₅H₂OH, and metaxylene diamine.

Chemical structures of compounds with at least one hydrophobic and at least two reactive functional components:

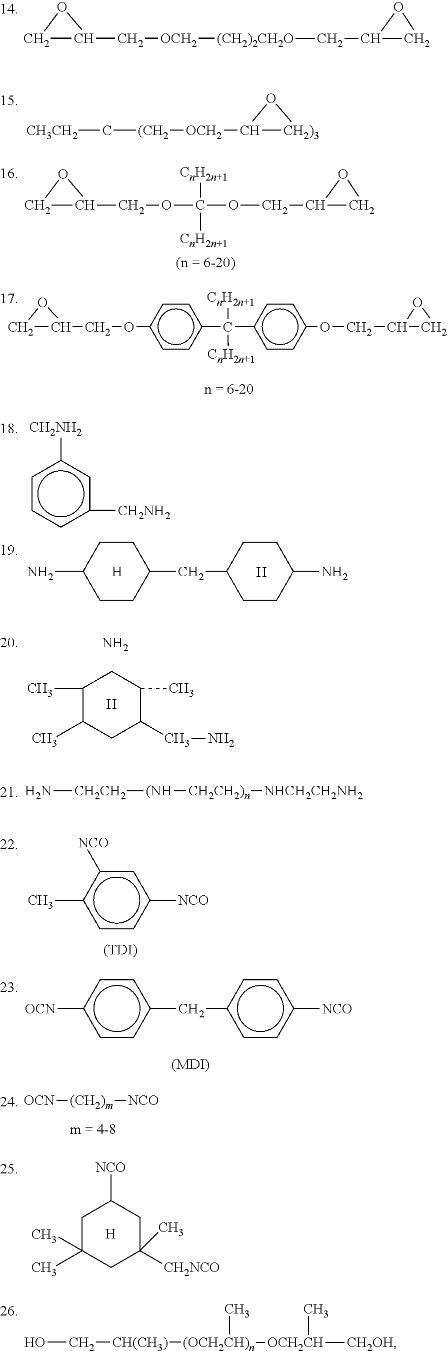

and combinations thereof.

The binder may comprise a catalyst or curing agent and the composition may be selected from the group consisting of chemical structures of compounds with at least one hydrophobic and at least two reactive functional components:

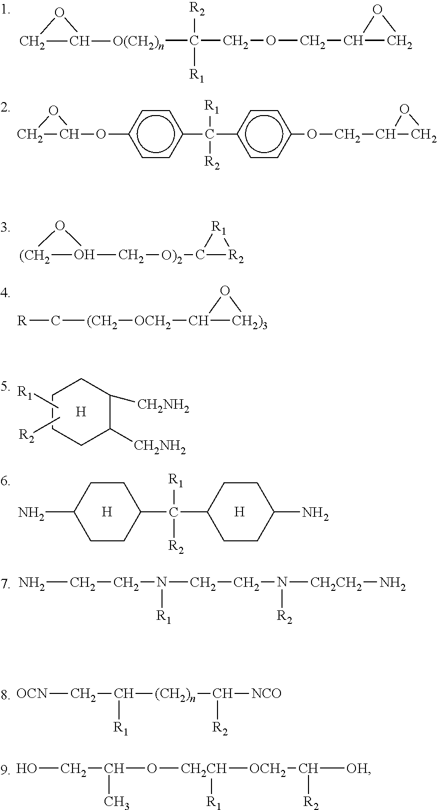

and combinations thereof.
R, R₁, R₂=C₆-C₂₀ (aliphatic, aromatic, cyclo aliphatic linear or branched)

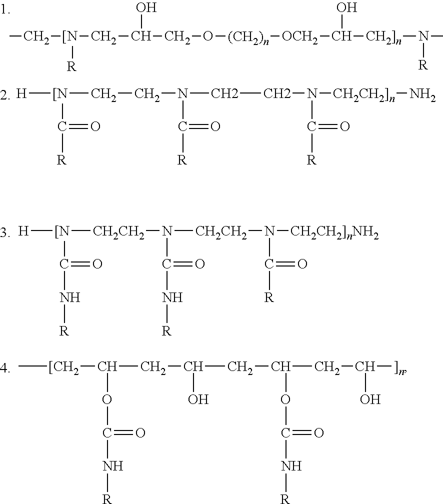

and combinations thereof.
n=50-100, R=C₆-C₂₀ [linear, branched, hydrocarbon chain, aliphatic, aromatic, acicyclic, alkylphenyl, chcloaliphatic, alkylcycloaliphatic. Structure of representative compounds that have Gemini structure Dag mitigating ingredients.

In yet a further illustrative example, a method of mitigating dag on hair comprises mixing tridecylglycidylether, fluorinated benzyl alcohol, adduct with tetraethylene pentamine or metaxylenediamine, and trimethylolpropane triglycidylether into the container to form a mixture; and applying the mixture to the hair.

There is thus provided a method for mitigating dag on hair. In at least one illustrative example, several types of active compounds or agents that may be transformed into active compounds, may be used to form a hydrophobic matrix with hair. For example, monofunctional epoxy, isocyanate, alcohols, amines as derivatives of fatty acids, long chain fatty acids such as cetyl alcohol, epoxy, cyanoacrylate, isocyanate, cetyl glycidyl ether, cresyl glycidyl ether, nonyl phenol glycidyl ethers, Gemini surfactants, and others as are known in the art, may become a part of a formed hydrophobic matrix. Many of these compounds may be commercially available and cost effective.

In at least one other illustrative example, cheap reactive organic hydrophobic agents that react and/or bond with keratin, cysteine and melanin and other amino acids may be used to form stable polymeric compositions thus transferring the cattle hair to be protective non-wet able hydrophobic compositions that prevents accumulation of dags whose composition is almost hydrophilic due to the presence of the partially digested cellulose and sugar residues.

In at least one other illustrative example, a method of mitigating dag is based on transferring the hair to be hydrophobic by reacting the hydrophobic active organic agents which include flexible long chain (C6-C20) linked to reactive groups such as epoxy, acrylate, isocyanate, and hydroxyl amine, which may instantly react within a reactive polymeric system to form chemical bonds with keratin, melanin and cysteine, the essential component of hair.

Keratin is a protein formed by the combination of 18 amino acids, among which cysteine being rich in sulphur plus other reactive functional groups that plays an important role in the cohesion of the hair. Cysteine represents the major active component of Keratin which can be used for changing the properties of hair from hydrophilic to hydrophobic. The coloring pigments such as melanin consist reactive functional groups can also be effective in this application.

These hair characteristics may be implemented by stylist to change the shape of human hair from curly to straight, or vice versa by oxidising this di sulphide bond at least removing one of the sulphide as H₂S, or H₂S₂ or reaction of the aldehyde groups and/or methylene glycol groups the active components of the hair straightening agent with active functional groups of the cysteine and melanin molecules.

In at least one additional illustrative example, the following reaction schemes may be used in the presently disclosed method to mitigate dag. Several types of reactions may take place at ambient temperature which may be implemented in present disclosure:

1—reactive hydrophobic active agents consisting epoxy active groups react photocatalytically in the visible light forming strong bonding with the cysteine, melanin and keratine the major hair components and/or in the presence of amine hardeners, several types of visible light photocatalysts are available.

2—Reactive hydrophobic primary amine long chain hydrocarbons are mixed with epoxy terminated elastomeric oily resins, standard hardener, mixed well then sprayed, the product polymerise quickly forming strong bonding with hair components. The elastic epoxy resins have strong adhesion strength with hair compositions.

3—cyanoacrylate group that can polymerised easily adopting the same reaction scheme well known with cyanoacrylate commercial adhesive, the cyano acrylate group is very reactive in the presence of humidity or any other active hydrogen containing compounds such as hair, human skin, cellulosic tissues and others forming strong bonding with the cysteine, melanin and keratine the major hair components.

4—Reactive hydrophobic reagents consisting isocyanate groups and the polyurethane starting material plus the catalysts and cocatalyst that react instantly and form strong bonding with hair composition this type of reaction is familiar in polyurethane adhesive technology.

5—long chain fatty alcohols which are excellent and cheap hydrophobic reagents react as composition within the polyurethane starting material plus the catalysts and cocatalyst that react instantly and form strong bonding with hair composition this type of reaction is familiar in polyurethane adhesive technology.

6—Molecular Gemini surfactants and molecular conventional surfactants that consist at least one hydroxyl group, or amino group and one or more of the hydrophobic chains are excellent and cheap reactive hydrophobic reagents react as composition within the polyurethane starting material plus the catalysts and cocatalyst that react instantly and form strong bonding with hair composition this type of reaction is familiar in polyurethane adhesive technology.

Spraying systems may be designed to be sprayed from distance to the cattle. The sprayed reactive super hydrophobic reagents or pre polymer may react and or bond with the keratin composition. Several type of reactive hydrophobic reagents previously presented may be sprayed and then polymerised in situe to form strong bonding with hair composition.

The presently disclosed method to mitigate dag may be non-stressing to the cattle, easy to apply by spraying, ambient temperature curing, nontoxic active ingredients, catalytic curing or visible light photo curing. These compounds may be relatively inexpensive as compared to silicon base hydrophobic reagents and fluorinated super hydrophobic reagents. For example, the cost of these reactive hydrophobic may be relatively low at about $5-8/l for the commercially available products which may be enough to treat 3-4 cattle.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of mitigating dag on hair, the method comprising:
applying a monofunctional compound comprising a hydrophobic tail and a reactive head to hair;
applying a difunctional compound to the hair, the difunctional compound having a first functional component and a second functional component; and
wherein the applying of the difunctional compound enables the first functional component to react with the reactive head of the monofunctional compound, the second functional component to form a matrix in situ with the hair, and the hydrophobic tail of the monofunctional compound to extend from the hair, the hydrophobic tail imparting its hydrophobicity to the hair to mitigate the dag.

2. The method of claim 1, comprising mixing the monofunctional compound and the difunctional compound prior to applying to the hair.

3. The method of claim 1, wherein the reactive head has at least one constituent selected from the group consisting of epoxy, acrylate, isocyanate, cyanoacrylate, amine, hydroxyl amine, alcohol, cetyl glycidyl ether, cresyl glycidyl ether, nonyl phenol glycidyl ether, n-butyl glycidyl ether, aliphatic glycidyl ether, 2-ethylhexyl glycidyl ether, phenyl glycidyl ether, o-cresyl glycidyl ether, p-tertiary butyl phenyl glycidyl ether, 3-alkyl phenol glycidyl ether, o-phenyl phenol glycidyl ether, benzyl glycidyl ether, and combinations thereof.

4. The method of claim 1, wherein the difunctional compound has at least one constituent selected from the group consisting of epoxy, acrylate, isocyanate, cyanoacrylate, amine, hydroxyl amine, alcohol, cetyl glycidyl ether, cresyl glycidyl ether, nonyl phenol glycidyl ether, n-butyl glycidyl ether, aliphatic glycidyl ether, 2-ethylhexyl glycidyl ether, phenyl glycidyl ether, o-cresyl glycidyl ether, p-tertiary butyl phenyl glycidyl ether, 3-alkyl phenol glycidyl ether, o-phenyl phenol glycidyl ether, benzyl glycidyl ether, and combinations thereof.

5. The method of claim 1, wherein the monofunctional compound comprises a constituent selected from the group consisting of: 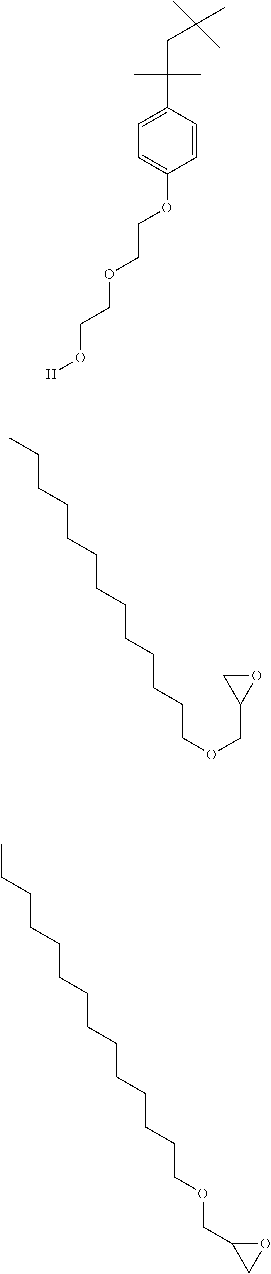 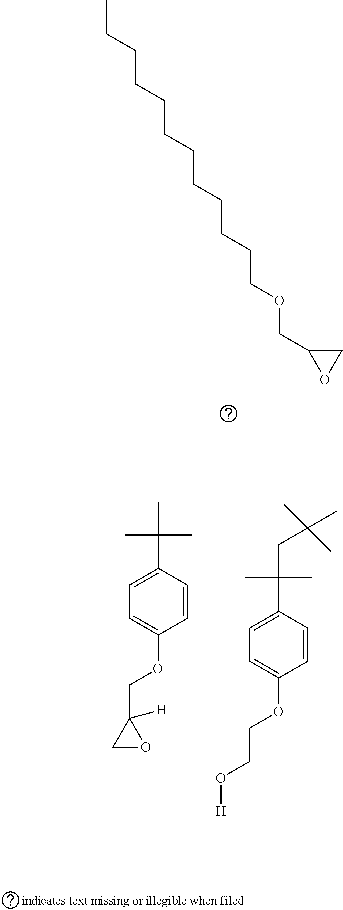 [SMILES: CCCCCCCCCCCCOCC1CO1 ; CC(C)(C)c1ccc(OCC2CO2)cc1 ; CC(C)(C)CC(C)(C)c1ccc(OCCO)cc1]
and combinations thereof.

6. The method of claim 1, wherein the monofunctional compound comprises a Gemini surfactant.

7. The method of claim 6, wherein the Gemini surfactant is represented by

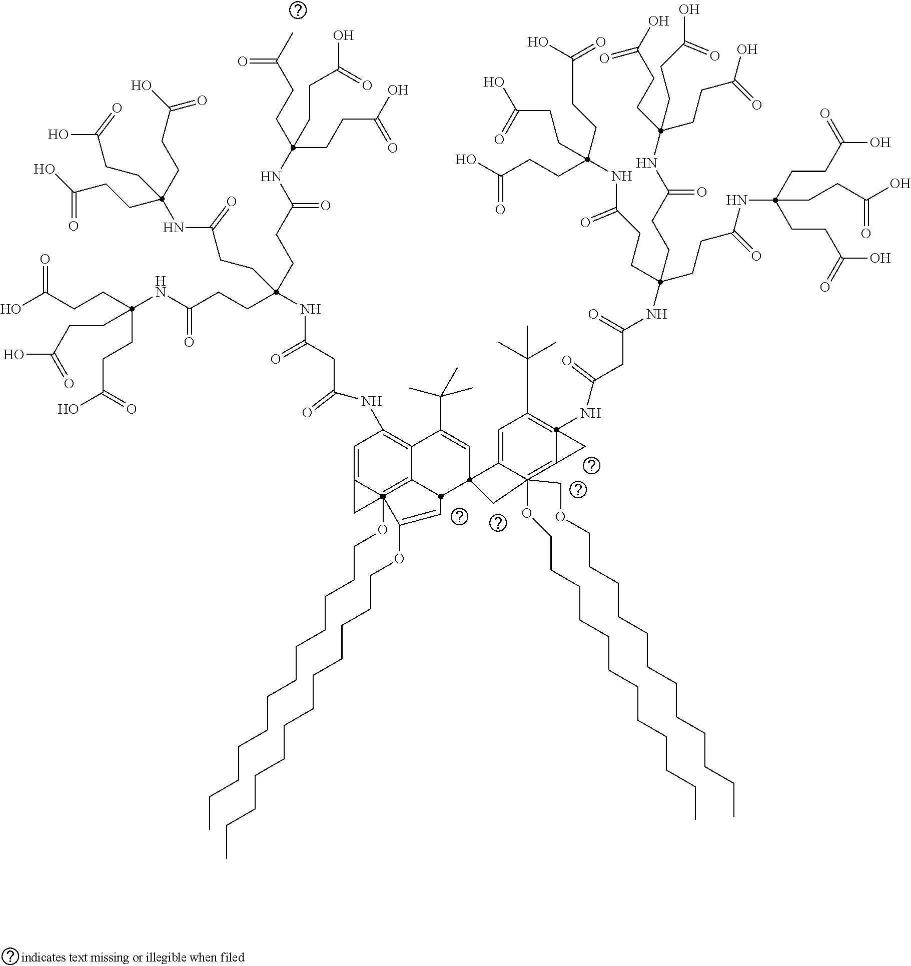

8. The method of claim 1, wherein the monofunctional compound comprises a flexible long hydrocarbon chain having between 6 and 20 carbon atoms.

9. The method of claim 1, wherein the monofunctional compound and the difunctional compound are photocatalytically reactive.

10. The method of claim 9, further comprising exposing the hair to light.

11. The method of claim 1, further comprising applying a hardener to the hair.

12. The method of claim 1, wherein the monofunctional compound is selected from the group consisting of:
CₙH₂ₙ₊₁—OH, CₙH₂ₙ₊₁—SH, CₙH₂ₙ₊₁—COOH, CₙH₂ₙ₊₁—NH₂, 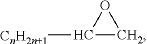
CₙH₂ₙ₊₁—OCH₂—CH₂,
CₙH₂ₙ₊₁—NCO, 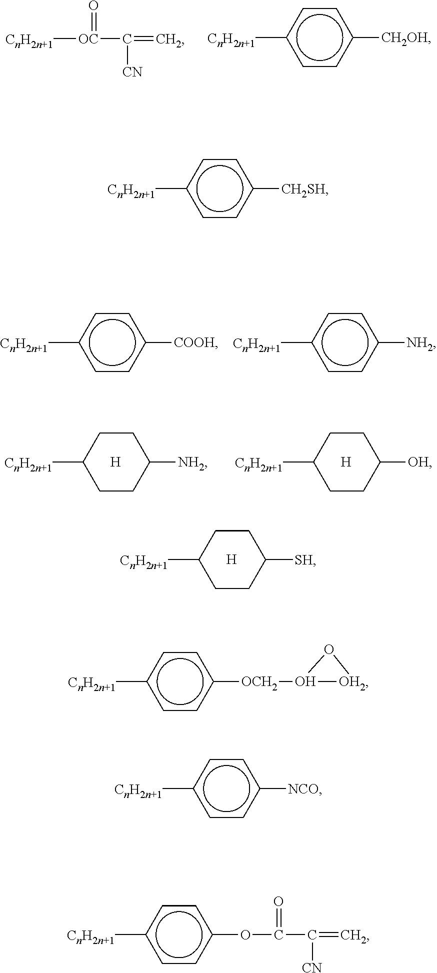
(CₙH₂ₙ₊₁)₃N,
(CₙH₂ₙ₊₁—COO)₂Zn⁺⁺,
(CₙH₂ₙ₊₁—COO)₂Ca⁺⁺, and combinations thereof, wherein n=6-20 (linear or branched).

13. The method of claim 1, wherein the monofunctional compound is selected from the group consisting of: CₙF₂ₙ₊₁—OH, CₙF₂ₙ₊₁—SH, CₙF₂ₙ₊₁—COOH, CₙH₂ₙ₊₁—NH₂, 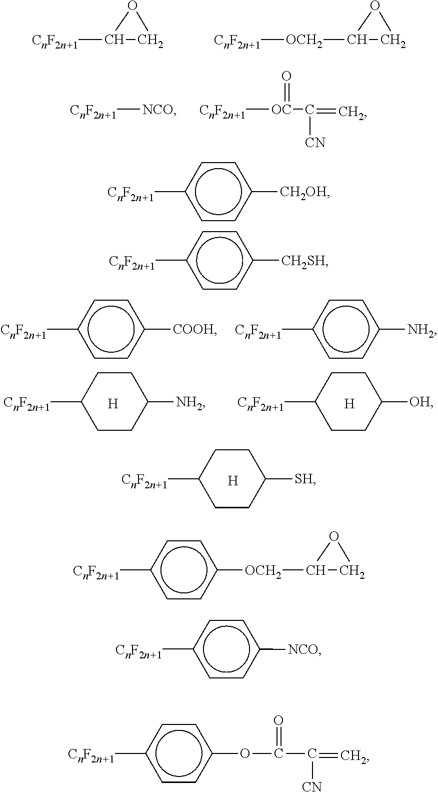
(CₙF₂ₙ₊₁)₃N, (CₙF₂ₙ₊₁—COO)₂Zn⁺⁺, (CₙF₂ₙ₊₁—COO)₂Ca⁺⁺, and combinations thereof, wherein n=6-20 (linear or branched).

14. The method of claim 1, wherein the monofunctional compound has a constituent selected from the group consisting of: stearate hydrophobic chain, linear chain aliphatic glycidyl ether C₁₂-C₁₄OCH₂CH(O)CH₂, linear chain aliphatic glycidyl ether; C₈-C₁₀OCH₂CH(O)CH₂, oleyl amine; C₁₉H₃₈NH₂, nonylphenylmonoglycidylether, C₉H₁₉C₆H₄OCH₂CH(O)CH₂, t-Butylphenylmonoglycidylether, C₄H₉C₆H₄OCH₂CH(O)CH₂, penta fluoro benzylalcohol C₇F₅H₂OH, and metaxylene diamine.

15. The method of claim 1, wherein the monofunctional compound is selected from the group consisting of: Cyanoacrylates, 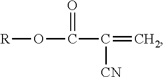
Glycidyl ethers, 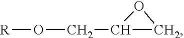
Caprolactam derivatives,
Caprolactone derivatives, 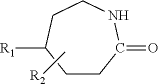
Polyurea, 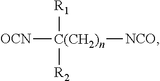
wherein n=2-8, 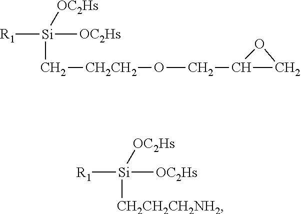
and combinations thereof,
wherein R, R1, R2=C6-C20 linear, branched, cydoaliphatic, alkyl phenyl, hydrocarbon chain, aliphatic, aromatic, cycloaliphatic, alkyl phenyl, alkyl cycloaliphatic.

16. The method of claim 1, wherein the difunctional compound has a constituent selected from the group consisting of: 1,4 butandiol di glycidylether, 1,1,1-triglycidylethertrimethylolpropane, neopentyldiglycidylether, stearic acid, oilyl amine, isophorone diamine, tetraethylene pentamine, polypropylene diamine, and bisphenol-A diglycidylether containing o-cresylmonoglycidyl ether.

17. The method of claim 1, wherein the monofunctional compound is C₁₃-monoglycidyl ether and the difunctional compound is butanediol diglycidyl ether.

18. The method of claim 1, further comprising adding a hardener to the hair.

19. A method of mitigating dag on hair, the method comprising:
applying a compound having a linking group and a hydrophobic material to the hair;
the hydrophobic material having at least one of an aliphatic group, an aromatic group, and an aliphatic aromatic group with a chain having C₆-C₂₀;
applying a binder to the hair; and
wherein the binder bonds with the hair and links with the linking group and enables the hydrophobic material to form a matrix in situ with the hair and impart its hydrophobicity to the hair.

20. The method of claim 19, wherein the hydrophobic material is selected from the group consisting of stearate hydrophobic chain, linear chain aliphatic glycidyl ether, C₁₂-C₁₄OCH₂CH(O)CH₂, linear chain aliphatic glycidyl ether, C₈-C₁₀OCH₂CH(O)CH₂, oleyl amine, C₁₉H₃₈NH₂, nonylphenylmonoglycidylether, C₉H₁₉ C₆H₄OCH₂CH(O)CH₂, t-butylphenylmonoglycidylether, C₄H₉C₆H₄OCH₂CH(O)CH₂, penta fluoro benzylalcohol C₇F₅H₂OH, metaxylene diamine, and combinations thereof.

21. The method of claim 19, wherein the binder is selected from the group consisting of 1,4 butandiol di glycidylether, 1,1,1-triglycidylethertrimethylolpropane, neopentyldiglycidylether, stearic acid, oilyl amine, isophorone diamine, tetraethylene pentamine, polypropylene diamine, bisphenol-A diglycidylether containing o-cresylmonoglycidyl ether, and combinations thereof.
